(19) United States
(12) Patent Application Publication
(10) Pub. No.: US 2014/0271822 A1
(43) Pub. Date: Sep. 18, 2014

(54) MODIFIED DOCETAXEL LIPOSOME FORMULATIONS

(71) Applicant: Mallinckrodt LLC, Hazelwood, MO (US)

(72) Inventors: William McGhee, Fenton, MO (US); James Blackledge, St. Louis, MO (US); Margaret Grapperhaus, Troy, IL (US)

(73) Assignee: Mallinckrodt LLC, Hazelwood, MO (US)

(21) Appl. No.: 14/208,324

(22) Filed: Mar. 13, 2014

Related U.S. Application Data
(60) Provisional application No. 61/779,902, filed on Mar. 13, 2013.

(51) Int. Cl. A61K 47/48; A61K 31/337; A61K 31/496; A61K 9/127

(52) U.S. Cl. CPC A61K 47/48815; A61K 9/1277; A61K 31/337; A61K 9/127; A61K 31/496
USPC 424/450; 514/449; 514/254.11

(57) ABSTRACT

The present invention provides compositions for the treatment of cancer. The compositions include liposomes containing a phosphatidylcholine lipid, a sterol, a PEG-lipid, and a taxane. The PEG-lipid constitutes from about 2 to about 8 mol % of the lipids in the liposome. The taxane is docetaxel esterified at the 2′-O position with a heterocyclyl-(C₂₋₅alkanoic acid). Methods for preparation of liposomal taxanes and treatment of cancer with liposomal taxanes are also disclosed.

### CROSS-REFERENCES TO RELATED APPLICATIONS

This application claims the benefit of priority of U.S. Provisional Application Ser. No. 61/779,902, filed Mar. 13, 2013, the content of which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Taxotere® (docetaxel) and Taxol® (paclitaxel) are the most widely prescribed anticancer drugs on the market, and are associated with a number of pharmacological and toxicological concerns, including highly variable (docetaxel) and non-linear (paclitaxel) pharmacokinetics, serious hypersensitivity reactions associated with the formulation vehicle (Cremophor E L, Tween 80), and dose-limiting myelosuppression and neurotoxicity. In the case of Taxotere®, the large variability in pharmacokinetics causes significant variability in toxicity and efficacy, as well as hematological toxicity correlated with systemic exposure to the unbound drug. In addition, since the therapeutic activity of taxanes increases with the duration of tumor cell drug exposure, the dose-limiting toxicity of commercial taxane formulations substantially limits their therapeutic potential. Resistance to the drugs due to causes such as up-regulation of protein transporter pumps by cancer cells can further complicate taxane-based therapies. As such, there exists a need for taxane-based chemotherapeutics with decreased toxicity and improved efficacy. The present invention addresses this and other needs.

### BRIEF SUMMARY OF THE INVENTION

In a first aspect, the present invention provides a composition for the treatment of cancer. The composition includes a liposome containing a phosphatidylcholine lipid, a sterol, a poly(ethylene glycol)-phospholipid conjugate (PEG-lipid), and a taxane or a pharmaceutically acceptable salt thereof. The taxane is docetaxel esterified at the 2′-O-position with a heterocyclyl-(C₂₋₅alkanoic acid), and the PEG-lipid constitutes 2-8 mol % of the total lipids in the liposome.

In a second aspect, the invention provides a method for preparing a liposomal taxane. The method includes: a) forming a first liposome having a lipid bilayer including a phosphatidylcholine lipid and a sterol, wherein the lipid bilayer encapsulates an interior compartment comprising an aqueous solution; b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoic acid); and c) forming a mixture containing the loaded liposome and a PEG-lipid under conditions sufficient to allow insertion of the PEG-lipid into the lipid bilayer.

In a third aspect, the invention provides a method for treating cancer. The method includes administering to a subject in need thereof the liposomal taxane of the invention.

### BRIEF DESCRIPTION OF THE DRAWINGS

FIG. 1 shows the clearance of TD-1 (A) and docetaxel (B) from plasma following administration of TD-1, TD-1 liposomes, and PEGylated TD-1 liposomes to mice bearing PC3 xenografts.

FIG. 2 shows the clearance of TD-1 from plasma following administration of PEGylated TD-1 liposomes to mice bearing A549 xenografts. Data are represented as mean+standard error of three mice or as the mean or single value if less than three mice.

FIG. 3 shows the levels of TD-1 (A) and docetaxel (B) in tumors following administration of TD-1, TD-1 liposomes and PEGylated TD-1 liposomes to mice bearing PC3 xenografts. Data are represented as mean±standard error of three mice.

FIG. 4 shows the levels of TD-1 (A) and docetaxel (B) in tumors following administration of PEGylated TD-1 liposomes and docetaxel to mice bearing A549 human NSCLC xenografts. Data are represented as mean±standard error of three mice or as the mean or single value if less than three mice.

FIG. 5 shows the levels of TD-1 in tissue following administration of 40 mg/kg (A) and 144 mg/kg (B) PEGylated TD-1 liposomes to mice bearing A549 human NSCLC xenografts. Data are represented as mean±standard error of three mice or as the mean or single value if less than three mice.

FIG. 6 shows the levels of docetaxel in tissue following administration of 40 mg/kg (A) and 144 mg/kg (B) PEGylated TD-1 liposomes to mice bearing A549 human NSCLC xenografts. Data are represented as mean+standard error of three mice or as the mean or single value if less than three mice.

FIG. 7(A) shows the antitumor effect of PEGylated TD-1 liposomes and docetaxel against human A253 (Head & Neck) tumor xenografts in athymic nude mice. Data are represented as mean±standard error (n=5-10). On day 31 post treatment, PEGylated TD-1 liposomes (90 mg/kg) treated mice have significantly smaller tumors than the saline (control) or docetaxel (30 mg/kg) treated mice, *, p<0.05, Newman-Keuls post hoc test following a one-way ANOVA. FIG. 7(B) shows a Kaplan-Meier survival plot of athymic nude mice bearing A253 (Head & Neck) xenograft tumors treated with PEGylated TD-1 liposomes, docetaxel or saline. PEGylated TD-1 liposomes (90 mg/kg) increased survival significantly greater than docetaxel and control, *, p<0.05, Mantel-Cox, log-rank test. Each group started with 10 female mice bearing tumors.

FIG. 8(A) shows the antitumor effect of PEGylated TD-1 liposomes and docetaxel against human A549 NSCLC tumor xenografts in athymic nude mice. Data are represented as mean+standard error (n=5-10). PEGylated TD-1 liposomes (90 mg/kg) significantly inhibited tumor growth compared to control or docetaxel (10, 20, and 30 mg/kg) on day 70 post treatment, *, p<0.05, ANOVA followed by Neuman-Keuls post hoc test. FIG. 8(B) shows a Kaplan-Meier survival plot of mice bearing A549 NSCLC xenograft tumors treated with PEGylated TD-1 liposomes, docetaxel or saline. Each group started with 10 female mice bearing tumors.

FIG. 9(A) shows the antitumor effect of PEGylated TD-1 liposomes and docetaxel against A549 human NSCLC tumor xenografts in nude mice. Test articles were administered on days 0 and 21. Administration of PEGylated TD-1 liposomes (60 & 90 mg/kg) and docetaxel (18 & 27 mg/kg) resulted in significantly smaller tumors than saline 37 days after initial treatment, *, p<0.05. Treatment with PEGylated TD-1 liposomes (60 & 90 mg/kg) resulted in significantly smaller tumors than docetaxel (18 & 27 mg/kg) at comparably tolerated doses on days 37 and 56 post treatment, #, p<0.05. One-way ANOVA followed by a Newman-Keuls post hoc test. Data are represented as mean±standard error of five to ten mice. FIG. 9(B) shows a Kaplan-Meier survival plot of athymic nude mice bearing A549 NSCLC xenograft tumors treated with PEGylated TD-1 liposomes, docetaxel or saline. All dose levels of PEGylated TD-1 liposomes and docetaxel increased survival significantly compared to saline, p<0.05, Mantel-Cox, log-rank test. Each group started with 10 female mice bearing tumors.

FIG. 10(A) shows the antitumor effect of TD-1 liposomes, PEGylated TD-1 liposomes, and docetaxel against human PC3 (prostate) tumor xenografts in athymic nude mice. All treatment groups exhibited significantly smaller tumors than saline 36 days following a single IV administration. Treatment with PEGylated TD-1 liposomes at 19 mg/kg caused significantly smaller tumors than the equitoxic dose of docetaxel (9 mg/kg) and TD-1 liposomes (30 mg/kg), *, p<0.05. PEGylated TD-1 liposomes (38 mg/kg) caused smaller tumors than docetaxel (18 mg/kg) at comparably tolerated doses on day 79 post treatment, #, p<0.05. One-way ANOVA followed by a Newman-Keuls post hoc test. Data are represented as mean of three to six mice. FIG. 10(B) shows a Kaplan-Meier survival plot of athymic nude mice bearing human PC3 (prostate) xenograft tumors treated with TD-1 liposomes, PEGylated TD-1 liposomes, docetaxel, or saline. Docetaxel treatment at 18 and 27 mg/kg and all treatment doses of TD-1 liposomes and PEGylated TD-1 liposomes increased survival significantly more than saline, p<0.05, Mantel-Cox, log-rank test. Each group started with 5 to 6 male mice bearing tumors.

FIG. 11(A) shows the antitumor effect of PEGylated TD-1 liposomes and docetaxel against MDA-MB-435/PTK7 (human breast) tumor xenografts in athymic nude mice. Median tumor volume (mm³) over time is shown after a single IV administration of test articles. Data are represented as median of four to eight mice. FIG. 11(B) shows a Kaplan-Meier survival plot showing percent survival of athymic nude mice bearing MDA-MB-435/PTK7 (human breast) xenograft tumors treated with a single administration of docetaxel, PEGylated TD-1 liposomes, or saline. Each group started with 8 female mice bearing tumors.

FIG. 12(A) shows the antitumor effect of PEGylated TD-1 liposomes and docetaxel against HT1080/PTK7 human fibrosarcoma tumor xenografts in athymic nude mice. Mean tumor volume (mm³) over time is shown after a single IV administration of docetaxel, PEGylated TD-1 liposomes, or saline. Treatment with PEGylated TD-1 liposomes (30, 60 & 90 mg/kg) and docetaxel (27 mg/kg) treatment caused significantly smaller tumors than saline on day 14 post treatment, *, p<0.05. Administration of PEGylated TD-1 liposomes (60 & 90 mg/kg) resulted in significantly smaller tumors than docetaxel (18 & 27 mg/kg) at corresponding equitoxic doses on day 21 post treatment,**, p<0.05. Administration of PEGylated TD-1 liposomes (90 mg/kg) resulted in significantly smaller tumors than docetaxel (27 mg/kg) at a comparably tolerated doses on day 30 post treatment, #, p<0.05. One-way ANOVA followed by a Newman-Keuls post hoc test. Data are represented as mean+standard error of five to ten mice. FIG. 12(B) shows a Kaplan-Meier survival plot of athymic nude mice bearing HT1080/PTK7 human fibrosarcoma xenograft tumors treated with docetaxel, PEGylated TD-1 liposomes, or saline. All doses levels of PEGylated TD-1 liposomes increased survival significantly greater than saline, *, p<0.05, and 90 mg/kg PEGylated TD-1 liposomes increased survival significantly greater than docetaxel (all dose levels), #, p<0.05, Mantel-Cox, log-rank test. Each group started with 10 female mice bearing tumors.

FIG. 13(A) Antitumor effect of PEGylated TD-1 liposomes and docetaxel against A431 human epidermoid tumor xenografts in athymic nude mice. Mean tumor volume (mm³) over time is shown after a single IV administration of PEGylated TD-1 liposomes, docetaxel or saline. All dose levels of PEGylated TD-1 liposomes and docetaxel caused significantly smaller tumors than saline on day 7 post treatment. PEGylated TD-1 liposomes (60 m/kg) caused significantly smaller tumors than treatment with either 20 or 30 mg/kg docetaxel, *, p<0.05. On day 17 post dose, groups treated with PEGylated TD-1 liposomes (60 and 90 mg/kg) exhibited significantly smaller tumors than docetaxel (20 mg/kg), #, p<0.05. One-way ANOVA followed by a Newman-Keuls post hoc test. Data are represented as mean+standard error of four to eight mice. FIG. 13(B) Kaplan-Meier survival plot showing percent survival of athymic nude mice bearing A431 human (epidermoid) xenograft tumors treated with PEGylated TD-1 liposomes, docetaxel, or saline. All dose levels of PEGylated TD-1 liposomes increased survival significantly more than saline and docetaxel (20 and 30 mg/kg), p<0.05, Mantel-Cox, log-rank test. Each group started with 8 female mice bearing tumors.

FIGS. 14a, 14b, and 14c provide a table of compositions evaluated to develop the claimed compositions and methods. The ratios provided in the Description column are the initial ratios for preparing a first liposome (prior to loading a taxane as described herein and prior to adding a PEG-lipid). The percentages of PC (phosphatidylcholine lipid), Chol (cholesterol) and DSPE-PEG2000 are provided in mol % following assembly of the final composition.

### DETAILED DESCRIPTION OF THE INVENTION

### I. General

The present invention provides novel liposomal taxanes, as well as a multi-step, one-pot method for encapsulation of taxanes in liposomes and subsequent incorporation of polyethylene glycol)-functionalized lipids into the liposomes. The liposomal taxanes prepared by the methods described herein demonstrate several advantages including increases in shelf stability, in vivo circulation time, and in vivo efficacy. The liposomal taxanes are useful for the treatment of cancer as described herein.

### II. Definitions

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size), and small unilamellar vesicles (SUVs; from about 20 nm to about 200 nm in size).

As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6-24 carbon atoms. Phosphatidylcholine lipids can include synthetic and naturally-derived 1,2-diacyl-sn-glycero-3-phosphocholines.

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl)tetracyclo[8.7.0.0^{2,7}.0^{11,15}]heptacos-7-en-5-ol; Chemical Abstracts Services Registry No. 57-88-5).

As used herein, the term “PEG-lipid” refers to a poly(ethylene glycol) polymer covalently bound to a hydrophobic or amphipilic lipid moiety. The lipid moiety can include fats, waxes, steroids, fat-soluble vitamins, monoglycerides, diglycerides, phospholipids, and sphingolipids. Preferred PEG-lipids include diacyl-phosphatidylethanolamine-N-[methoxy(polyethene glycol)]s and N-acyl-sphingosine-1-{succinyl[methoxy(polyethylene glycol)]}s. The molecular weight of the PEG in the PEG-lipid is generally from about 500 to about 5000 Daltons (Da; g/mol). The PEG in the PEG-lipid can have a linear or branched structure.

As used herein, the term “taxane” refers to a compound having a structural skeleton similar to diterpene natural products, also called taxanes, initially isolated from yew trees (genus Taxus). Taxanes are generally characterized by a fused 6/8/6 tricyclic carbon backbone, and the group includes natural products and synthetic derivatives. Examples of taxanes include, but are not limited to, paclitaxel, docetaxel, and cabazitaxel. Certain taxanes of the present invention include ester moieties at the 2′ hydroxyl group of the 3-phenypropionate sidechain that extends from the tricyclic taxane core.

As used herein, the term “heterocyclyl” refers to a saturated or unsaturated ring system having from 3 to 12 ring members and from 1 to 4 heteroatoms of N, O and S. The heteroatoms can also be oxidized, such as, but not limited to, —S(O)— and —S(O)₂—. Heterocyclyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. Heterocyclyl includes, but is not limited to, 4-methylpiperazinyl, morpholino, and piperidinyl.

As used herein the term “alkanoic acid” refers to a carboxylic acid containing 2-5 carbon atoms. The alkanoic acids may be linear or branched. Examples of alkanoic acids include, but are not limited to, acetic acid, propionic acid, and butanoic acid.

As used herein, the terms “molar percentage” and “mol %” refer to the number of a moles of a given lipid component of a liposome divided by the total number of moles of all lipid components. Unless explicitly stated, the amounts of active agents, diluents, or other components are not included when calculating the mol % for a lipid component of a liposome.

As used herein, the term “loading” refers to effecting the accumulation of a taxane in a liposome. The taxane can be encapsulated in the aqueous interior of the liposome, or it can be embedded in the lipid bilayer. Liposomes can be passively loaded, wherein the taxane is included in the solutions used during liposome preparation. Alternatively, liposomes can be remotely loaded by establishing a chemical gradient (e.g., a pH or ion gradient) across the liposome bilayer, causing migration of the taxane from the aqueous exterior to the liposome interior.

As used herein, the term “insertion” refers to the embedding of a lipid component into a liposome bilayer. In general, an amphiphilic lipid such as a PEG-lipid is transferred from solution to the bilayer due to van der Waals interactions between the hydrophobic portion of the amphiphilic lipid and the hydrophobic interior of the bilayer.

As used herein, the term “composition” refers to a product comprising the specified ingredients in the specified amounts, as well as any product which results, directly or indirectly, from combination of the specified ingredients in the specified amounts. Pharmaceutical compositions of the present invention generally contain a liposomal taxane as described herein and a pharmaceutically acceptable carrier, diluent, or excipient. By “pharmaceutically acceptable,” it is meant that the carrier, diluent, or excipient must be compatible with the other ingredients of the formulation and non-deleterious to the recipient thereof.

As used herein, the term “cancer” refers to conditions including human cancers and carcinomas, sarcomas, adenocarcinomas, lymphomas, leukemias, and solid and lymphoid cancers. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma.

As used herein, the terms “treat”, “treating” and “treatment” refer to any indicia of success in the treatment or amelioration of a cancer or a symptom of cancer, including any objective or subjective parameter such as abatement; remission; diminishing of symptoms or making the cancer or cancer symptom more tolerable to the patient. The treatment or amelioration of symptoms can be based on any objective or subjective parameter, including, e.g., the result of a physical examination or clinical test.

As used herein, the terms “administer,” “administered,” or “administering” refer to methods of administering the liposome compositions of the present invention. The liposome compositions of the present invention can be administered in a variety of ways, including parenterally, intravenously, intradermally, intramuscularly, or intraperitoneally. The liposome compositions can also be administered as part of a composition or formulation.

As used herein, the term “subject” refers to any mammal, in particular a human, at any stage of life.

As used herein, the term “about” indicates a close range around a numerical value when used to modify that specific value. If “X” were the value, for example, “about X” would indicate a value from 0.9X to 1.1X, and more preferably, a value from 0.95X to 1.05X. Any reference to “about X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X.

### III. Embodiments of the Invention

In a first aspect, the present invention provides a composition for the treatment of cancer. The composition includes a liposome containing a phosphatidylcholine lipid, a sterol, a PEG-lipid, and a taxane or a pharmaceutically acceptable salt thereof. The taxane is esterified with a heterocyclyl-(C₂₋₅alkanoic acid), and the PEG-lipid constitutes 2-8 mol % of the total lipids in the liposome.

### Taxanes

In some embodiments, the taxane is a compound according to Formula I, or a pharmaceutically acceptable salt thereof.

[chemical structure]

For compounds of Formula I, R¹ is selected from phenyl and t-butoxy; R² is selected from H, acetyl and methyl; R³ is selected from H, 4-(4-methylpiperazin-1-yl)-butanoyl and methyl; and R⁴ is selected from H and heterocyclyl-C₂₋₅alkanoyl. At least one of R³ and R⁴ is other than H.

Compounds of Formula I are useful as chemotherapeutic agents for the treatment of various cancers, including breast cancer, ovarian cancer, and lung cancer. Formula I encompasses paclitaxel derivatives, wherein R¹ is phenyl. Paclitaxel itself can be obtained by various methods including total chemical synthesis as well as semisynthetic methods employing 10-deacetylbaccatin III (10-DAB; Formula II, below). 10-DAB can be isolated from Pacific and European yew trees (Taxus brevifolia and Taxus baccata, respectively) and can be used as a starting material for preparation of paclitaxel and other taxanes including, but not limited to, docetaxel (i.e., R¹=t-butoxy; R², R³, R⁴=H) and cabazitaxel according to known methods. Taxane preparation via semisynthetic methods are contemplated for use in the present invention in addition to taxane preparation via total synthesis.

[chemical structure]

As described above, the use of taxanes—including paclitaxel and docetaxel—for cancer therapy can be limited by low bioavailability due to inadequate solubility, as well as by high toxicity. Various strategies have been employed to remedy these drawbacks. For example, derivatization of the taxane skeleton at the C7 and C10 functional groups of the tricylic core, or at the C2′ hydroxyl group of the C13 sidechain, with moieties of varying polarity can be used to alter the bioavailability of taxane-base drugs (see, for example, U.S. Pat. No. 6,482,850; U.S. Pat. No. 6,541,508; U.S. Pat. No. 5,608,087; and U.S. Pat. No. 5,824,701).

Incorporation of a taxane into liposomes can improve bioavailability and reduce the toxicity of the taxane. In the present invention, modification of the taxane skeleton with weak base moieties can facilitate the active loading of otherwise poorly water-soluble taxanes into the aqueous interior of a liposome. In general, the weak base moiety can include an ionizable amino group, such as an N-methyl-piperazino group, a morpholino group, a piperidino group, a bis-piperidino group or a dimethylamino group. In some embodiments, the weak base moiety is an N-methyl-piperazino group.

A taxane can be derivatized in a region that is not essential for the intended therapeutic activity such that the activity of the derivative is substantially equivalent to that of the free drug. For example, in some aspects, the weak base derivative comprises the taxane docetaxel derivatized at the 7-OH group of the baccatin skeleton. In some embodiments, docetaxel derivatives are provided which are derivatized at the 2′-OH group which is essential for docetaxel activity.

Accordingly, some embodiments of the present invention provide liposomes containing a taxane or a pharmaceutically acceptable salt thereof, wherein the taxane is docetaxel esterified at the 2′-O-position with a heterocyclyl-(C₂₋₅alkanoic acid) (i.e., the taxane is a compound of Formula I wherein R¹ is t-butoxy; R² is H; R³ is H; and R⁴ is heterocyclyl-C₂₋₅alkanoyl). In some embodiments, the heterocyclyl-(C₂₋₅alkanoic acid) is selected from 5-(4-methylpiperazin-1-yl)-pentanoic acid, 4-(4-methylpiperazin-1-yl)-butanoic acid, 3-(4-methylpiperazin-1-yl)-propionic acid, 2-(4-methylpiperazin-1-yl)-ethanoic acid, 5-morpholino-pentanoic acid, 4-morpholino-butanoic acid, 3-morpholino-propionic acid, 2-morpholino-ethanoic acid, 5-(piperidin-1-yl)pentanoic acid, 4-(piperidin-1-yl)butanoic acid, 3-(piperidin-1-yl)propionic acid, and 2-(piperidin-1-yl)ethanoic acid. In some embodiments, the heterocyclyl-(C₂₋₅alkanoic acid) is 4-(4-methylpiperazin-1-yl)-butanoic acid.

### Liposomes

The liposomes of the present invention can contain any suitable lipid, including cationic lipids, zwitterionic lipids, neutral lipids, or anionic lipids as described above. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

In general, the liposomes of the present invention contain at least one phosphatidylcholine lipid (PC). Suitable phosphatidylcholine lipids include saturated PCs and unsaturated PCs.

Examples of saturated PCs include 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (OSPC).

Lipid extracts, such as egg PC, heart extract, brain extract, liver extract, soy PC, and hydrogenated soy PC(HSPC) are also useful in the present invention.

The compositions provided herein will, in some embodiments, consist essentially of PC/cholesterol mixtures (with an added taxane and PEG-lipid as described below). In some embodiments, the liposome compositions will consist essentially of a phosphatidylcholine lipid or mixture of phosphatidylcholine lipids, with cholesterol, a PEG-lipid and a taxane. In still other embodiments, the liposome compositions will consist essentially of a single type of phosphatidylcholine lipid, with cholesterol, a PEG-lipid and a taxane. In some embodiments, when a single type of phosphatidylcholine lipid is used, it is selected from DOPC, DSPC, HSPC, DPPC, POPC and SOPC.

In some embodiments, the phosphatidylcholine lipid is selected from the group consisting of DPPC, DSPC, HSPC, and mixtures thereof. In some embodiments, the compositions of the present invention include liposomes containing 50-65 mol % of a phosphatidylcholine lipid or mixture of phosphatidylcholine lipids or 45-70 mol % of a phosphatidylcholine lipid or mixture of phosphatidylcholine lipids. The liposomes can contain, for example, 50, 51, 52, 53, 54, 55, 56, 57, 58, 59, 60, 61, 62, 63, 64 or 65 mol % phosphatidylcholine. In some embodiments, the liposomes contain about 55 mol % phosphatidylcholine. In some embodiments, the liposomes contain about 53 mol % phosphatidylcholine.

Other suitable phospholipids, generally used in low amounts or in amounts less than the phosphatidylcholine lipids, include phosphatidic acids (PAs), phosphatidylethanolamines (PEs), phosphatidylglycerols (PGs), phosphatidylserine (PSs), and phosphatidylinositol (PIs). Examples of phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPE), dielaidoylphosphoethanolamine (transDOPE), and cardiolipin.

In some embodiments, phospholipids can include reactive functional groups for further derivatization. Examples of such reactive lipids include, but are not limited to, dioleoylphosphatidylethanolamine-4-(N-maleimidomethyl)-cyclohexane-1-carboxylate (DOPE-mal) and dipalmitoylphosphatidylethanolamine-N-succinyl (succinyl-PE).

Liposomes of the present invention can contain steroids, characterized by the presence of a fused, tetracyclic gonane ring system. Examples of steroids include, but are not limited to, cholic acid, progesterone, cortisone, aldosterone, testosterone, dehydroepiandrosterone, and sterols such as estradiol and cholesterol. Synthetic steroids and derivatives thereof are also contemplated for use in the present invention.

In general, the liposomes contain at least one sterol. In some embodiments, the sterol is cholesterol (i.e., 2,15-dimethyl-14-(1,5-dimethylhexyl)tetracyclo[8.7.0.0^{2,7}.0^{11,15}]heptacos-7-en-5-op. In some embodiments, the liposomes can contain about 30-50 mol % of cholesterol, or about 30-45 mol % of cholesterol. The liposomes can contain, for example, 30, 31, 32, 33, 34, 35, 36, 37, 38, 39, 40, 41, 42, 43, 44, or 45 mol % cholesterol. In some embodiments, the liposomes contain 30-40 mol % cholesterol. In some embodiments, the liposomes contain 40-45 mol % cholesterol. In some embodiments, the liposomes contain 45 mol % cholesterol. In some embodiments, the liposomes contain 44 mol % cholesterol.

The liposomes of the present invention can include any suitable poly(ethylene glycol)-lipid derivative (PEG-lipid). In some embodiments, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The poly(ethylene glycol) can have a molecular weight of, for example, 750 Da, 1000 Da, 2000 Da, or 5000 Da. In some embodiments, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000). In some embodiments, the PEG-lipid is DSPE-PEG-2000.

In general, the compositions of the present invention include liposomes containing 2-8 mol % of the PEG-lipid. The liposomes can contain, for example, 2, 3, 4, 5, 6, 7, or 8 mol % PEG-lipid. In some embodiments, the liposomes contain 2-6 mol % PEG-lipid. In some embodiments, the liposomes contain 3 mol % PEG-lipid. In some embodiments, the liposomes contain 3 mol % DSPE-PEG-2000.

The liposomes of the present invention can also include some amounts of cationic lipids—which are generally amounts lower than the amount of phosphatidylcholine lipid. Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N—(N′,N′-dimethylaminoethane)carbamoyl] cholesterol (DC-Chol), dimethyldioctadecylammonium (DDAB) and N,N-dimethyl-2,3-dioleyloxy)propylamine (DODMA).

In some embodiments of the present invention, the liposome includes from about 50 mol % to about 70 mol % of DSPC and from about 25 mol % to about 45 mol % of cholesterol. In some embodiments, the liposome includes about 53 mol % of DSPC, about 44 mol % of cholesterol, and about 3 mol % of DSPE-PEG-2000. In some embodiments, the liposome includes about 66 mol % of DSPC, about 30 mol % of cholesterol, and about 4 mol % of DSPE-PEG-2000.

### Diagnostic Agents

The liposomes of the present invention may also contain diagnostic agents. A diagnostic agent used in the present invention can include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The diagnostic agents can be associated with the therapeutic liposome in a variety of ways, including for example being embedded or encapsulated in the liposome.

In some embodiments, a diagnostic agent can include chelators that bind to metal ions to be used for a variety of diagnostic imaging techniques. Exemplary chelators include but are not limited to ethylenediaminetetraacetic acid (EDTA), [4-(1,4,8,11-tetraazacyclotetradec-1-yl)methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ₇₅Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃—IDA, and ^{99m}Tc(CO)₃-triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging. 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to gadopentetic acid, gadoteric acid, gadodiamide, gadolinium, gadoteridol, mangafodipir, gadoversetamide, ferric ammonium citrate, gadobenic acid, gadobutrol, or gadoxetic acid. Superparamagnetic agents can include but are not limited to superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexyl, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexyl, iopentol, ioversol, iobitridol, iodixanol, iotrolan and iosimenol.

### Targeting Agents

In some cases, liposome accumulation at a target site may be due to the enhanced permeability and retention characteristics of certain tissues such as cancer tissues. Accumulation in such a manner often results in part because of liposome size and may not require special targeting functionality. In other cases, the liposomes of the present invention can also include a targeting agent. Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. In some embodiments, the targeting component can be specific to only one target, such as a receptor. Suitable targets can include but are not limited to a nucleic acid, such as a DNA, RNA, or modified derivatives thereof. Suitable targets can also include but are not limited to a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell.

In certain embodiments, a targeting agent can include a target ligand (e.g., an RGD-containing peptide), a small molecule mimic of a target ligand (e.g., a peptide mimetic ligand), or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

### Methods for Preparing Liposomal Taxane

In a second aspect, the invention provides methods for preparing a liposomal taxane. Liposomes can be prepared and loaded with taxanes using a number of techniques that are known to those of skill in the art. Lipid vesicles can be prepared, for example, by hydrating a dried lipid film (prepared via evaporation of a mixture of the lipid and an organic solvent in a suitable vessel) with water or an aqueous buffer. Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous buffer. Unilamellar vesicles can be formed from MLVs via sonication or extrusion through membranes with defined pore sizes. Encapsulation of a taxane can be conducted by including the drug in the aqueous solution used for film hydration or lipid dilution during MLV formation. Taxanes can also be encapsulated in pre-formed vesicles using “remote loading” techniques. Remote loading includes the establishment of a pH- or ion-gradient on either side of the vesicle membrane, which drives the taxane from the exterior solution to the interior of the vesicle.

Accordingly, some embodiments of the present invention provide a method for preparing a liposomal taxane including: a) forming a first liposome having a lipid bilayer including a phosphatidylcholine lipid and a sterol, wherein the lipid bilayer encapsulates an interior containing an aqueous solution; b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoyl) group; and c) incorporating the PEG-lipid into the lipid bilayer.

The taxanes and lipids used in the methods of the invention are generally as described above. However, the route to the liposomal taxane will depend in part on the identity of the specific taxane and lipids and the quantities and combinations that are used. For example, the taxane can be encapsulated in vesicles at various stages of liposome preparation. In some embodiments, the first liposome is formed such that the lipid bilayer comprises DSPC and cholesterol, and the DSPC:cholesterol ratio is about 55:45 (mol:mol). In some embodiments, the first liposome is formed such that the lipid bilayer comprises DSPC and cholesterol, and the DSPC:cholesterol ratio is about 70:30 (mol:mol). In some embodiments, the interior of the first liposome contains aqueous ammonium sulfate buffer. Loading the first liposomes can include forming an aqueous solution containing the first liposome and the taxane or pharmaceutically acceptable salt thereof under conditions sufficient to allow accumulation of the taxane in the interior compartment of the first liposome.

Loading conditions generally include a higher ammonium sulfate concentration in the interior of the first liposome than in the exterior aqueous solution. In some embodiments, the loading step is conducted at a temperature above the gel-to-fluid phase transition temperature (Tₘ) of one or more of the lipid components in the liposomes. The loading can be conducted, for example, at about 50, about 55, about 60, about 65, or at about 70° C. In some embodiments, the loading step is conducted at a temperature of from about 50° C. to about 70° C. Loading can be conducted using any suitable amount of the taxane. In general, the taxane is used in an amount such that the ratio of the combined weight of the phosphatidylcholine and the sterol in the liposome to the weight of the taxane is from about 1:0.01 to about 1:1. The ratio of the combined phosphatidylcholine/sterol to the weight of the taxane can be, for example, about 1:0.01, about 1:0.05, about 1:0.10, about 1:0.15, about 1:0.20, about 1:0.25, about 1:0.30, about 1:0.35, about 1:0.40, about 1:0.45, about 1:0.50, about 1:0.55, about 1:0.60, about 1:0.65, about 1:0.70, about 1:0.75, about 1:0.80, about 1:0.85, about 1:0.90, about 1:0.95, or about 1:1. In some embodiments, the loading step is conducted such that the ratio of the combined weight of the phosphatidylcholine and the sterol to the weight of the taxane is from about 1:0.01 to about 1:1. In some embodiments, the ratio of the combined weight of the phosphatidylcholine and the sterol to the weight of the taxane is from about 1:0.05 to about 1:0.5. In some embodiments, the ratio of the combined weight of the phosphatidylcholine and the sterol to the weight of the taxane is about 1:0.2. The loading step can be conducted for any amount of time that is sufficient to allow accumulation of the taxane in the liposome interior at a desired level.

The PEG-lipid can also be incorporated into lipid vesicles at various stages of the liposome preparation. For example, MLVs containing a PEG-lipid can be prepared prior to loading with a taxane. Alternatively, a PEG-lipid can be inserted into a lipid bilayer after loading of a vesicle with a taxane. The PEG-lipid can be inserted into MLVs prior to extrusion of SUVs, or the PEG-lipid can be inserted into pre-formed SUVs.

Accordingly, some embodiments of the invention provide a method for preparing a liposomal taxane wherein the method includes: a) forming a first liposome having a lipid bilayer including a phosphatidylcholine lipid and a sterol, wherein the lipid bilayer encapsulates an interior compartment comprising an aqueous solution; b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoyl) group; and c) forming a mixture containing the loaded liposome and a poly(ethylene glycol)-phospholipid conjugate (PEG-lipid) under conditions sufficient to allow insertion of the PEG-lipid into the lipid bilayer.

In some embodiments, the insertion of the PEG-lipid is conducted at a temperature of from about 35-70° C. The loading can be conducted, for example, at about 35, about 40, about 45, about 50, about 55, about 60, about 65, or at about 70° C. In some embodiments, insertion of the PEG-lipid is conducted at a temperature of from about 50° C. to about 55° C. Insertion can be conducted using any suitable amount of the PEG-lipid. In general, the PEG-lipid is used in an amount such that the ratio of the combined number of moles of the phosphatidylcholine and the sterol to the number of moles of the PEG-lipid is from about 1000:1 to about 20:1. The molar ratio of the combined phosphatidylcholine/sterol to PEG lipid can be, for example, about 1000:1, about 950:1, about 900:1, about 850:1, about 800:1, about 750:1, about 700:1, about 650:1, about 600:1, about 550:1, about 500:1, about 450:1, about 400:1, about 350:1, about 300:1, about 250:1, about 200:1, about 150:1, about 100:1, about 50:1, or about 20:1. In some embodiments, the loading step is conducted such that the ratio of combined phosphatidylcholine and sterol to PEG-lipid is from about 1000:1 to about 20:1 (mol:mol). In some embodiments, the ratio of the combined phosphatidylcholine and sterol to the PEG-lipid is from about 100:1 to about 20:1 (mol:mol). In some embodiments, the ratio of the combined phosphatidylcholine and sterol to the PEG-lipid is from about 35:1 (mol:mol) to about 25:1 (mol:mol). In some embodiments, the ratio of the combined phosphatidylcholine and sterol to the PEG-lipid is about 33:1 (mol:mol). In some embodiments, the ratio of the combined phosphatidylcholine and sterol to the PEG-lipid is about 27:1 (mol:mol).

A number of additional preparative techniques known to those of skill in the art can be included in the methods of the invention. Liposomes can be exchanged into various buffers by techniques including dialysis, size exclusion chromatography, diafiltration, and ultrafiltration. Buffer exchange can be used to remove unencapsulated taxanes and other unwanted soluble materials from the compositions. Aqueous buffers and certain organic solvents can be removed from the liposomes via lyophilization. In some embodiments, the methods of the invention include exchanging the liposomal taxane from the mixture in step c) to an aqueous solution that is substantially free of unencapsulated taxane and uninserted PEG-lipid. In some embodiments, the methods include lyophilizing the liposomal taxane.

### Methods of Treating Cancer

In another aspect, the invention provides a method of treating cancer. The method includes administering to a subject in need thereof a composition containing a liposomal taxane as described above. In therapeutic use for the treatment of cancer, the liposome compositions of the present invention can be administered such that the initial dosage of the taxane ranges from about 0.001 mg/kg to about 1000 mg/kg daily. A daily dose of about 0.01-500 mg/kg, or about 0.1-200 mg/kg, or about 1-100 mg/kg, or about 10-50 mg/kg, or about 10 mg/kg, or about 5 mg/kg, or about 2.5 mg/kg, or about 1 mg/kg can be used.

The dosages may be varied depending upon the requirements of the patient, the type and severity of the cancer being treated, and the liposome composition being employed. For example, dosages can be empirically determined considering the type and stage of cancer diagnosed in a particular patient. The dose administered to a patient should be sufficient to affect a beneficial therapeutic response in the patient over time. The size of the dose will also be determined by the existence, nature, and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under circumstances is reached. For convenience, the total daily dosage may be divided and administered in portions during the day, if desired.

The methods described herein apply especially to solid tumor cancers (solid tumors), which are cancers of organs and tissue (as opposed to hematological malignancies), and ideally epithelial cancers. Examples of solid tumor cancers include bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer and renal cancer. In one group of embodiments, the solid tumor cancer suitable for treatment according to the methods of the invention are selected from CRC, breast and prostate cancer. In another group of embodiments, the methods of the invention apply to treatment of hematological malignancies, including for example multiple myeloma, T-cell lymphoma, B-cell lymphoma, Hodgkins disease, non-Hodgkins lymphoma, acute myeloid leukemia, and chronic myelogenous leukemia.

The comopositions may be administered alone in the methods of the invention, or in combination with other therapeutic agents. The additional agents can be anticancer agents or cytotoxic agents including, but not limited to, avastin, doxorubicin, cisplatin, oxaliplatin, carboplatin, 5-fluorouracil, gemcitibine or other taxanes. Additional anti-cancer agents can include, but are not limited to, 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

### IV. Examples

### Example 1

### Preparation of Liposomal Taxane

### Buffer and Reagent Preparation

300 mM Sucrose Dialysis Solution Preparation.

102.69 g sucrose was weighed and added to a 1 L volumetric flask. The flask was filled three-quarters full DI water and mixed by shaking until solids were dissolved. DI water was added at room temperature to bring the sucrose to the desired concentration and mixed by repeatedly inverting the capped flask. The solution was filtered through a 0.2 μm 47 mm nylon membrane by vacuum and stored at 2-5° C.

350 mM Ammonium Sulfate Buffer Solution Preparation.

23.13 g ammonium sulfate was weighed and added to a class A 500 mL volumetric flask. The flask was filled three-quarters full DI water and mixed by shaking until solids were dissolved. DI water was added at room temperature to bring the ammonium sulfate to the desired concentration and mixed by repeatedly inverting the capped flask. The solution was filtered through a 0.2 μm 47 mm nylon membrane by vacuum and stored at 2-5° C.

350 mM Ammonium Sulfate/100 mM Sucrose Buffer Solution Preparation.

34.24 g sucrose and 46.24 g ammonium sulfate were weighed and added to a 1 L class A volumetric flask. The flask was filled three-quarters full DI water and mixed by shaking until solids were dissolved. DI water was added at room temperature to bring the solution to the desired concentration and mixed by repeatedly inverting the capped flask. The solution was filtered through a 0.2 μm 47 mm nylon membrane by vacuum and stored at 2-5° C.

Lipid Solvation.

DSPC (1.785 g) and cholesterol (0.715 g) were weighed in clean glass weighing funnels. The materials were charged into a clean 1 L round bottom flask. 15 mL of ethanol were added using a class A volumetric pipet at room temperature. The round bottom flask was connected to a rotary evaporator water bath at 60° C. The flask was rotated at 150 RPM and 60° C. in the bath without vacuum until all materials were completely dissolved (about 30 minutes). The lipids solution was maintain at 60° C. temperature after solvation. 85 mL of ammonium sulfate/sucrose was measured in a class A graduated cylinder, covered with parafilm, and heated to 60° C. using a water bath.

### Liposome Preparation

The 1 L round bottom flask was removed from the rotary evaporator. The heated 85 mL of ammonium sulfate/sucrose was discharged into the flask while vigorously swirling. The mixture was rotated in the flask on the rotary evaporator bath at 60° C. for 30 minutes. The flask was then removed and extrusion was initiated immediately.

Extrusion.

Four glass serum bottles and stoppers were prepared by rinsing three times with ethanol and drying with UHP nitrogen. The bottles were capped until sample addition. A 100 mL extruder was assembled with one drain disc and two 0.2 μm nucleopore membranes added to extruder filtration base. The extruder was decontaminated by completing a 100 mL pass of DI water heated to 70° C. The liposome solution was discharged from the 1 L flask into the extruder heated to 70° C. The liposomes were extruded and passed into a 250 mL glass beaker. The 200-nm membrane was replace with dual 100 nm membranes and the system was purged once into a clean 250 mL beaker. The extrusion was repeated 10 times using the clean beaker. The final liposome sample was collected in the cleaned serum bottle, capped, sealed, and cooled to room temperature. The liposomes were stored at 2-5° C.

Diafiltration.

A Spectrum KrossFlo Unit diafiltration apparatus was cleaned with 1 L 0.1 N NaOH heated to 95° C. at flow rate of 100 ml/min and a transmembrane pressure of 3 Psi. The flow was reversed after 500 mL was eluted, and the flow was continued for an additional 500 mL. The sample reservoir was filled and replaced at least three times, and the system was purged dry before rinsing. Dust and debris was cleaned from the tubing exterior with isopropanol wipes. A sterile, 0.1 μm 25 mm PVDF syringe filter was inserted into a GL45 media bottle cap for air intake filtration. The system was rinsed with 1 L of DI water at room temperature at 100 mL/min and 3 Psi transmembrane pressure. The sample reservoir was filled and replaced with DI water at least three times. The system was purged dry before purging the system with 300 mM sucrose at room temperature. The sample reservoir was emptied and rinsed three times with ethanol and three times with DI water (˜10 mL per rinse). The extruded liposome sample was added to the sample reservoir at room temperature.

The dialfitration was started using a 500 kDa cut-off mPES hollow fiber module, a pump rate of 100.0±1.0 mL/min, a TMP of 3.0±1.0 Psi, a Pp of −0.3≦0.0 Psi, and a Pf of 5.0±1.0 Psi. The diafiltration was continued until the filtrate volume reached approximately 30 times the retentate volume. The sample was removed from the reservoir and discharged into a clean serum bottle. The sample was filtered through a 0.2 μm 25 mm syringe filter into a clean serum bottle. The sample was then filtered through a 0.1 μm 25 mm sterile inorganic syringe filter into a clean serum bottle while disposing the first three eluted drops. The sample was capped, sealed, and stored at 2-5° C. Following extrusion, the sample was characterized in terms of particle size, pH, lipid concentration, and ammonium concentration.

Remote Loading of 2′O-4-(4-methylpiperazin-1-yl)-butanoyl-docetaxel (TD-1) and Insertion of DSPE-PEG-2000.

Remote Loading Procedure.

Docetaxel derivative TD-1 (386 mg, prepared as described in WO 2009/141738 A2) was weighed in a 500 mL 3-neck round bottom flask fitted with two rubber stoppers, an adaptor for a temperature controlling thermocouple, and a stir bar. TD-1 was dissolved in 190 mL of 10 mM acetate-buffered sucrose solution (pH 5.5), and the pH of the solution was adjusted to 5.5-5.6 using aqueous sodium hydroxide. The solution was heated to 65° C. using a heating mantle with moderate stirring.

To a second 500 mL round bottom flask was added the liposomal ammonium sulfate sample (1.932 g of total lipid). The liposomes were diluted with acetate-buffered sucrose to a final volume of 196 mL and the pH was adjusted to 5.5. The mixture was heated to 65° C. using the thermocouple-controlled heating mantle and poured into the solution of TD-1. Heating was continued for 15 minutes, and then the temperature was reduced to 55° C. A sample of the liposomes was collected for size and pH analysis.

Insertion of DSPE-PEG-2000.

DSPE-PEG-2000 (290 mg) was dissolved in 8 mL of acetate-buffered sucrose and added to the heated liposome solution. The mixture was maintained at 55° C. for 30 min. The heating mantle was removed, and the mixture was allowed to cool to ambient temperature. A sample of the liposomes was collected for size and pH analysis.

Diafiltration.

The diafiltration apparatus was equilibrated with 20 mM acetate/300 mM sucrose buffer as described above. 250 mL of the liposome mixture was added to the reservoir and concentrated via ultrafiltration to a total volume of about 50 mL. The remaining liposome mixture was added and concentrated to 50 mL. The ultrafiltrates were diafiltered against at least 15 volumes of 20 mM acetate/300 mM sucrose, pH 5.50. The liposomes were concentrated to 60 mL and sampled for size and pH analysis. Samples were analyzed for quantification of TD-1, docetaxel, DSPC, cholesterol, DSPE-PEG-2000 and lyso-DSPC. The final liposome preparation was stored in a clear serum vial with a butyl rubber stopper and crimped seal at 5° C.

Liposomes preparations prepared according to the above method were stored under varying conditions and analyzed in terms of particle size and drug release as summarized in Table 1. The liposomes were compared to non-PEGylated samples. PEGylation of the liposomes led to unexpected gains in liposome integrity, as assessed by the level of the drug observed to leak from the liposomes upon storage. Leakage of TD-1 from PEGylated liposomes upon freezing was reduced by nearly an order of magnitude with respect to non-PEGylated liposomes. Suprisingly, leakage of TD-1 from PEGylated liposomes upon storage at 5° C. was reduced by factor of over 22.

TABLE 1
PEGylated and Non-PEGylated Liposomes
Under Varying Storage Conditions

| | | | | Re- | | |
|---|---|---|---|---|---|---|
| | | | | leased | Particle | |
| Exam- | | | Temp. | Drug | Size | |
| ple | Lipid | Buffer | (° C.) | (%) | (vol. nm) | pDI |
| 1a | DSPC: Chol | 300 mM | 5 | 5.9 | 85 | 0.091 |
| | | Sucrose | | | | |
| 1b | DSPC: Chol | 300 mM | −20 | 12.3 | 146 | 0.268 |
| | | Sucrose | | | | |
| 1c | DSPC: Chol: | 300 mM | 5 | 0.27 | 109.8 | 0.038 |
| | DSPE-PEG | Sucrose | | | | |
| | (2000) | | | | | |
| 1d | DSPC: Chol: | 300 mM | −20 | 1.31 | 109.3 | 0.032 |
| | DSPE-PEG | Sucrose | | | | |
| | (2000) | | | | | |

### Example 2

### Control of PEG-Lipid Insertion into Liposomal Taxane Compositions

It has been found that the incorporation of DSPE-PEG(2000) as a thermal insertion step is best established after drug loading. Careful control of temperature and time for the insertion of DSPE-PEG(2000) was found to provide adequate PEGylation, with details from various lots given in Table 3. In all cases terminal sterilization of PEGylated TD-1 liposomes was carried out by filtration through 0.2 micron filters with careful control of all incoming raw materials.

Empty liposomes were prepared as described above for loading with TD-1 and insertion of PEG to form the final PEGylated TD-1 liposomes. The Tables below compare various parameters for lots of materials generated and the PEG insertion conditions used.

TABLE 2
Conditions for TD-1 Loading into Liposomes

| Batch # | 1 | 2 | 3 | 4 | 5 | 6 | 7 |
|---|---|---|---|---|---|---|---|
| TD-1 (g) | 26.05 | 25.43 | 25.41 | 25.50 | 25.53 | 28.61 | 23.73 |
| TD-1 | 5.1 | 5.1 | 5.1 | 5.1 | 5.1 | 5.1 | 5.1 |
| concentration in | | | | | | | |
| sucrose (mg/ml) | | | | | | | |
| TD-1 loading | 1.7 | 1.5 | 1.5 | 1.5 | 1.5 | 1.5 | 1.5 |
| concentration (mg/ml) | | | | | | | |
| Volume of LUVs | 2.84 | 2.36 | 2.18 | 2.76 | 2.57 | 2.05 | 2.38 |
| required (L) | | | | | | | |
| Volume of Suc/ | 5.37 | 7.04 | 7.31 | 7.13 | 7.06 | 8.95 | 7.38 |
| Acetate buffer | | | | | | | |
| (L) | | | | | | | |
| Volume of sucrose | 4.60 | 4.34 | 4.37 | 4.54 | 4.43 | 5.00 | 4.48 |
| solution for API (L) | | | | | | | |
| Volume of sucrose | 1.00 | 1.00 | 1.00 | 1.00 | 1.00 | 1.00 | 1.00 |
| solution for rinse (L) | | | | | | | |
| Total drug loading | 13.81 | 14.74 | 14.86 | 15.43 | 15.06 | 17 | 15.24 |
| reaction volume (L) | | | | | | | |
| Temperature of drug | 51.6 | 58.7 | 58.3 | 58.2 | 58.2 | 58.3 | 60.0 |
| solution (° C.) | | | | | | | |
| Temperature of | 62.0 | 61.5 | 62.9 | 60.9 | 59.0 | 59.5 | 61.7 |
| Diluted liposome | | | | | | | |
| (° C.) | | | | | | | |
| Loading Time at | 15.0 | 15.0 | 15.0 | 15.0 | 15.0 | 15.0 | 15.0 |
| 60° C. (mins) | | | | | | | |

TABLE 3
DSPE-PEG(2000) Thermal Insertion Parameters.

| Batch # | 1 | 2 | 3 | 4 | 5 | 6 | 7 |
|---|---|---|---|---|---|---|---|
| DSPE-PEG | 16.00 | 15.08 | 15.20 | 15.78 | 15.40 | 15.26 | 16.13 |
| (2000) Added | | | | | | | |
| (g) | | | | | | | |
| Thermal insertion | 62 | 50 | 55 | 55 | 54 | 54 | 52-56 |
| temperature (° C.) | | | | | | | |
| Thermal insertion | 30 | 30 | 30 | 60 | 60 | 60 | 60 |
| time (mins) | | | | | | | |
| Final volume (L) | 13.81 | 14.74 | 14.86 | 15.43 | 15.06 | 17.00 | 15.24 |

TABLE 4
Analysis of Liposomal TD-1 Produced Using Various Insertion Parameters

| Batch # | 1 | 2 | 3 | 4 | 5 | 6 | 7 |
|---|---|---|---|---|---|---|---|
| TD-1 loaded | 11.93 | 22.00 | 19.28 | 20.86 | 19.74 | 18.52 | 19.96 |
| (Free Base) (g) | | | | | | | |
| TD-1 added (g) | 26.05 | 25.43 | 25.41 | 25.5 | 25.53 | 28.61 | 22.86 |
| DSPE-PEG (2000) | 13.17 | 4.34 | 7.03 | 13.68 | 14.19 | 14.01 | 10.9 |
| inserted (g) | | | | | | | |
| DSPE-PEG | 16.37 | 15.41 | 15.52 | 16.14 | 15.71 | 15.51 | 15.58 |
| (added) (g) | | | | | | | |
| Drug to | 0.096 | 0.200 | 0.176 | 0.175 | 0.173 | 0.165 | 0.166 |
| lipid ratio | | | | | | | |
| Drug Loading | 45.8% | 86.5% | 75.9% | 81.8% | 77.3% | 64.7% | 87.0% |
| yield (%) | | | | | | | |
| PEG insertion | 80.5% | 28.2% | 45.3% | 84.8% | 90.3% | 90.3% | 67.6% |
| yield (%) | | | | | | | |

The major, significant variation in process parameters for the 7 lots described above occurred during the incorporation of DSPE-PEG(2000) into the drug loaded liposome. As indicated in Table 3 and Table 4, PEGylation depended upon the temperature and time used for insertion. Lower temperatures (e.g., 50° C.) or shorter time periods (e.g., 30 min hold time) led to lower DSPE-PEG(2000) in the drug product (e.g, lot 2 with 28.2% PEG incorporation and lot 3 with 45.3% PEG incorporation), while high temperatures (e.g., 62° C.) provided higher PEG incorporation (80.5%) at the sacrifice of drug substance encapsulation (45.8%) which leads to a lower drug to lipid ratio in the drug product. Heating to 55° C. for 60 min. was shown to provide good yields of both drug loading and PEG incorporation (65-87% and 68-90% respectively for the final 4 batches described in the Table).

### Example 3

### Biodistribution of Liposomal Taxane Derivative, Comparative Results

Two pharmacokinetic and tissue distribution studies have been completed in tumor bearing mice comparing PEGylated TD-1 liposomes with docetaxel.

Intravenous administration of the PEGylated TD-1 liposomes resulted in a systemic exposure to docetaxel 10 times greater than equivalent amounts of docetaxel injected as the free drug. Both the TD-1 and docetaxel accumulated in both PC3 and A549 tumors after intravenous injection of PEGylated TD-1 liposomes. The concentration of TD-1 and docetaxel increased slowly for up to 72 hours after dosing and remained in the tumor throughout the observation periods (up to 21 days). In contrast, intravenous injection of docetaxel resulted in high concentrations in the tumor initially which decreased over a seven day period and then fell below the levels of detection.

In addition to accumulating in tumor tissue, TD-1 and docetaxel also accumulated in the liver, spleen and kidney after the administration of PEGylated TD-1 liposomes. These tissues showed a similar biodistribution pattern as the tumor with slow uptake and stable prolonged residence times. In contrast, free docetaxel did not collect in these tissues and fell below the levels of detection within 24 hours of injection. Although docetaxel concentrations were detectable in lung tissue through 24 hours, analytical measurements failed to detect the presence of docetaxel in the skeletal muscle tissue.

A late increase in TD-1 and docetaxel concentrations in the tumor at the high dose of PEGylated TD-1 liposomes (144 mg/kg) was not consistent with the behavior found at the lower dose or in the other tissues and may be a calculation artifact due to long drug exposure in tumors that shrank significantly in size.

Encapsulation of TD-1 in both non-PEGylated and PEGylated liposomes increased the systemic exposure (AUC) to docetaxel compared to both the non-encapsulated TD-1 and docetaxel while producing a lower peak plasma concentration (Cₘₐₓ).

Pharmacokinetic investigations in mice demonstrate benefits in terms of greater and more sustained exposures to the active drug docetaxel within tumors, with lower peak blood levels. This suggests the possibility of enhanced anti-tumor activity in human patients without increased toxicity.

### Methods

Design.

The plasma pharmacokinetics and distribution were studied in male athymic nude mice each implanted subcutaneously with PC3 cells (human prostate cancer). Once tumors reached a volume of 100-300 mm³, animals were randomized into 5 groups. Each animal was given a single intravenous dose of docetaxel, unencapsulated TD-1, non-PEGylated TD-1 liposomes, or PEGylated TD-1 liposomes as shown in Table 5.

TABLE 5
Dosing assignments for nude mice bearing PC3 xenografts

Dose (mg/kg)

| Test Article | No. Animals | TD-1^{a} | Docetaxel^{b} |
|---|---|---|---|
| Docetaxel | 24 | 0 | 19 |
| TD-1 | 24 | 13.5 | 11 |
| TD-1 liposomes | 24 | 37.5 | 30 |
| PEGylated TD-1 | 24 | 37.5 | 30 |
| liposomes | | | |
| PEGylated TD-1 | 24 | 75 | 60 |
| liposomes | | | |

^{a}= Test article dose is expressed as mg/kg TD-1
^{b}= Test article dose is expressed as mg/kg docetaxel equivalent (TD-1/1.25 conversion factor)

Three animals were sacrificed at 5 minutes and 1, 4, 24, 48, 72, 120 and 168 hours post injection. Blood samples were taken for pharmacokinetic analysis at each time. Pharmacokinetic parameters of TD-1 and docetaxel were calculated using the Phoenix WinNonLin software by non-compartment analysis modeling.

### Results

The plasma concentration of TD-1 decreased with time, as shown in FIG. 1A. Compared with either form of the encapsulated drug, unencapsulated TD-1 demonstrated low systemic exposure (AUC), rapid clearance and a large volume of distribution (Table 6).

TABLE 6
Pharmacokinetics of TD-1 following administration
of TD-1, TD-1 liposomes and PEGylated TD-1
liposomes to nude mice bearing PC3 xenografts

| | | | PEGylated | PEGylated |
|---|---|---|---|---|
| | | TD-1 | TD-1 | TD-1 |
| Compound | TD-1 | liposomes | liposomes | liposomes |
| Dose (mg/kg)^{a} | 11 | 30 | 30 | 60 |
| AUC (μg · h/mL) | 5.7 | 20589 | 28156 | 42487 |
| AUC/Dose | 0.4 | 549 | 751 | 566 |
| Cₘₐₓ (μg/mL) | 5.0 | 833 | 1022 | 1805 |
| Cₘₐₓ/Dose | 0.4 | 22 | 27 | 24 |
| CL (mL/h/kg) | 2386 | 1.8 | 1.3 | 1.8 |
| t_{1/2} (h) | 7.6 | 6.6 | 9.7 | 12 |
| Vz (mL/kg) | 26010 | 17 | 19 | 30 |

^{a}All doses were given as the molar equivalent of docetaxel.

Although the doses of TD-1 were higher with the liposomal formulations (TD-1 liposomes and PEGylated TD-1 liposomes), an increase in dose from 11 to 30 mg/kg resulted in a 3600 to 4900 fold increase in the systemic exposure for the encapsulated TD-1. In addition, encapsulation of TD-1 slowed the clearance and restricted the volume of distribution compared to the non-encapsulated formulation. These data indicate that the liposomal forms of TD-1 remain in the plasma for a prolonged period of time. An increase in the PEGylated TD-1 liposomes dose from 30 to 60 mg/kg increased the Cₘₐₓ and systemic exposure to TD-1, but did not alter clearance, terminal half life or volume of distribution.

The plasma concentration of docetaxel decreased with time (FIG. 1B). Although stable under acidic or protected conditions (encapsulated), TD-1 readily hydrolyzes to form docetaxel under neutral pH and non-protected conditions. The non-encapsulated TD-1 and docetaxel exhibited similar docetaxel concentration-time curves with concentrations falling below the levels of detection after 48 hours. After the administration of encapsulated TD-1, docetaxel concentrations also fell but the rate of decrease was slowed compared to the free drugs. Quantifiable concentrations of docetaxel occurred through 120 and 168 hours after 30 and 60 mg/kg, respectively.

As free drugs, docetaxel and TD-1 generated plasma docetaxel concentrations having pharmacokinetic parameters of relatively small systemic exposures, rapid clearance and large volumes of distribution compared to TD-1 liposomes and PEGylated TD-1 liposomes (Table 7).

TABLE 7
Pharmacokinetics of docetaxel following administration
of TD-1, TD-1 liposomes and PEGylated TD-1
liposomes to nude mice bearing PC3 xenografts

| | | | | PEGylated | PEGylated |
|---|---|---|---|---|---|
| | | | TD-1 | TD-1 | TD-1 |
| Compound | Docetaxel | TD-1 | liposomes | liposomes | liposomes |
| Dose (mg/kg)^{a} | 19 | 11 | 30 | 30 | 60 |
| AUC | 13 | 4.1 | 116 | 113 | 463 |
| (μg · h/mL) | | | | | |
| AUC/Dose | 0.7 | 0.4 | 3.9 | 3.8 | 7.7 |
| Cₘₐₓ (μg/mL) | 13 | 1.4 | 3.1 | 2.9 | 32 |
| Cₘₐₓ/Dose | 0.7 | 0.1 | 0.1 | 0.1 | 0.5 |
| CL (mL/h/kg) | 1499 | 2591 | 259 | 266 | 130 |
| t_{1/2} (h) | 7.4 | 11 | 19 | 39 | 17 |
| Vz (mL/kg) | 16110 | 40905 | 6953 | 15058 | 3145 |

^{a}All doses were given as the molar equivalent of docetaxel.

Both docetaxel and non-encapsulated TD-1 displayed similar plasma docetaxel concentrations, which is consistent with conversion of TD-1 to docetaxel. The slower clearance, increased half life, and increased systemic exposure of docetaxel provided by PEGylated TD-1 liposomes indicates that the encapsulated TD-1 serves as a reservoir for continual release from the liposomes and conversion to docetaxel.

Pharmacokinetics in Mice with A549 Xenografts

The plasma pharmacokinetics and distribution were studied in female athymic nude mice each implanted subcutaneously with A549 cells (human non-small cell lung cancer). Once tumors reached a volume of 100-300 mm³, animals were randomized into 4 groups. Each animal was given a single intravenous dose of docetaxel or PEGylated TD-1 liposomes as shown in Table 8.

TABLE 8
Dosing assignments for nude mice
bearing A459 xenografts

| | | Dose (mg/kg) | |
|---|---|---|---|
| Test Article | No. Animals | TD-1 ^{a} | Docetaxel ^{b} |
| Docetaxel | 27 | 0 | 30 |
| Docetaxel | 27 | 0 | 50 |
| PEGylated TD-1 | 27 | 50 | 40 |
| liposomes | | | |
| PEGylated TD-1 | 27 | 180 | 144 |
| liposomes | | | |

^{a} Test article dose is expressed as mg/kg TD-1
^{b} Test article dose is expressed as mg/kg docetaxel equivalent (TD-1/1.25 conversion factor)

Three animals were sacrificed at 1, 4, 24, 72 (3 days), 168 (7 days), 216 (9 days), 336 (14 days), 432 (18 days) and 504 hours (21 days) post injection. Blood samples were taken for pharmacokinetic analysis at each time point. Pharmacokinetic parameters of TD-1 and docetaxel were calculated using the Phoenix WinNonLin software by non-compartment analysis modeling.

The plasma concentration of TD-1 decreased with time, as shown in FIG. 2. At a dose of 40 mg/kg, TD-1 concentrations remained above the limits of quantitation (0.025 μg/mL) through 168 hours after liposome administration; whereas, following a dose of 144 mg/kg, TD-1 was detected through the entire three week observation period after liposome administration. The Cₘₐₓ and systemic exposure (plasma AUC) to TD-1 increased with an increase in the dose of PEGylated TD-1 liposomes (Table 9).

TABLE 9
Pharmacokinetics of TD-1 following
administration of PEGylated TD-1 liposomes
to nude mice bearing A549 xenografts

| Dose (mg/kg) | 40 | 144 |
|---|---|---|
| t_{1/2} (h) | 10.5 | 10.5 |
| Cₘₐₓ (μg/mL) | 786 | 2907 |
| AUC_{∞} (μg · h/mL) | 20920 | 112682 |
| CL (mL/h/kg) | 2.4 | 1.6 |
| Vd (mL/kg) | 36 | 24 |

After iv injection of PEGylated TD-1 liposomes, plasma concentrations of docetaxel slowly decreased over time and remained above the limits of detection through three and seven days after doses of 40 and 144 mg/kg, respectively. In contrast, docetaxel, administered as the free drug, was detectable for only four hours. PEGylated TD-1 liposomes (40 mg/kg) exhibited Cₘₐₓ docetaxel concentrations similar to those resulting from the administration of docetaxel (50 mg/kg) itself but the exposure, in terms of AUC, was almost 10 times greater (Table 10).

TABLE 10
Pharmacokinetics of docetaxel following
administration of PEGylated TD-1 liposomes
to nude mice bearing A549 xenografts

| | | | PEGylated | |
|---|---|---|---|---|
| Compound | Docetaxel | | TD-1 liposomes | |
| Dose (mg/kg) | 30 | 50 | 40 | 144 |
| t_{1/2} (h) | * | * | 12 | 16 |
| Cₘₐₓ (μg/mL) | 2.7 | 8.6 | 10 | 36 |
| AUC_{∞} (μg · h/mL) | 8.8 | 27 | 267 | 1146 |
| CL (mL/h/kg) | * | * | 148 | 126 |
| Vd (mL/kg) | 16110 | 37187 | 2531 | 2848 |

* = Not calculable

The docetaxel derived from PEGylated TD-1 liposomes appeared to be restricted to a smaller volume of distribution compared to docetaxel administered as the free drug. The plasma concentration of docetaxel generated from PEGylated TD-1 liposomes was approximately 1% that of TD-1 measured in the blood through 3 days post dose.

Tissue Distribution in Mice with PC3 Xenografts

In addition to the plasma levels and pharmacokinetic calculations described above, tissue distribution was also evaluated. Tissues harvested from each animal described in Table 5 and frozen before analysis included: tumor, liver, spleen, and kidney. Tissues from mice treated with docetaxel were analyzed for docetaxel levels. Tissues from mice treated with TD-1, TD-1 liposomes and PEGylated TD-1 liposomes were analyzed for both docetaxel and TD-1 levels.

For the liposomal formulations, the concentration of TD-1 initially increased in PC3 tumor tissue after which the concentrations remained fairly constant through the 168 hour observation time period, (FIG. 3A). In contrast, the tumor concentration of TD-1 after administration of non-encapsulated TD-1 fell in concentration through approximately 24 hours and remained at very low concentrations through the remainder of the observation period.

The concentration of docetaxel in the tumor slowly increased over 48 to 72 hours after the administration of TD-1 liposomes and PEGylated TD-1 liposomes and then remained relatively stable through the remainder of the observation period (FIG. 3B). After the administration of non-encapsulated TD-1 the tumor concentration of docetaxel increased quickly and remained elevated through the observation period. Administration of docetaxel as a free drug resulted in the rapid onset of high concentrations of docetaxel in the tumor. Although dosed at approximately ⅔ the encapsulated dose, administration of free docetaxel resulted in higher earlier concentrations than the encapsulated formulations and similar concentrations at 120 and 168 hours after injection.

After the administration of docetaxel, non-encapsulated TD-1, non-PEGylated TD-1 liposomes and PEGylated TD-1 liposomes, the liver, spleen and kidney contained both docetaxel and TD-1 (Table 11). The spleen tended to have a greater exposure (AUC) to docetaxel than the liver and kidney for all formulations tested. The liver, spleen, and kidney had less exposure to docetaxel after the administration of PEGylated TD-1 liposomes compared to the non-PEGylated TD-1 liposomes. The data are consistent with less uptake of PEGylated liposomes by the organs of clearance.

TABLE 11
Tissue distribution of docetaxel in nude mice bearing
PC3 xenografts following treatment with docetaxel,
TD-1 liposomes or PEGylated TD-1 liposomes

| | | | TD-1 | PEGylated TD- |
|---|---|---|---|---|
| Compound | Docetaxel | TD-1 | liposomes | 1 liposomes |
| Dose (mg/kg)^{a} | 19 | 11 | 30 | 30 |
| Tumor AUC (μg · h/g) | 896 | 314 | 469 | 355 |
| Liver AUC (μg · h/g) | 50 | —^{b} | 324 | 181 |
| Spleen AUC (μg · h/g) | 94 | —^{b} | 708 | 640 |
| Kidney AUC (μg · h/g) | 73 | —^{b} | 380 | 227 |

^{a}All doses are given as docetaxel molar equivalents.
^{b}Samples not assayed.

Tissue Distribution in Mice with A549 Xenografts

In addition to the plasma levels and pharmacokinetic calculations, an assessment of tissue distribution was done in A549 human NSCLC tumor bearing mice after the administration of PEGylated TD-1 liposomes (as in Table 8). TD-1 accumulated in the A549 tumors for an extended period of time (FIG. 4A). The concentration of TD-1 increased slowly through the first 24 hours after injection. After 24 hours, concentrations of TD-1 tended to drift downward with time at the low dose. At the high dose, concentrations remained somewhat stable through approximately 14 days post dose and then tended to increase but the variability also increased. The concentration of TD-1 remained above the lower limits of quantitation (2.0 μg/g) through the 21 day observation period.

Similar to administration of unencapsulated TD-1, administration of PEGylated TD-1 liposomes resulted in increasing concentrations of docetaxel in the A549 tumors through the first 7 days for low dose (40 mg/kg) and through 9 days for the high dose (144 mg/kg). After the initial peak, docetaxel concentrations decreased slightly and then remained stable through the remainder of the 21 day observation period following the low dose (FIG. 4B). After the high dose of PEGylated TD-1 liposomes, concentrations of docetaxel decreased slightly and again increased 18 and 21 days after dosing. In contrast, intravenous injection of docetaxel peaked immediately after injection and then decreased with time falling below the levels of quantitation (1.0 μg/g) after nine days.

At comparable doses, PEGylated TD-1 liposomes (40 mg/kg) exhibited a tumor exposure (AUC) of docetaxel 3.9 times greater than the administration of docetaxel (50 mg/kg) itself (Table 12).

TABLE 12
Levels of docetaxel in tissue following administration
of docetaxel or PEGylated TD-1 liposomes to nude
mice bearing A549 xenografts

| | | | PEGylated | |
|---|---|---|---|---|
| Compound | Docetaxel | | TD-1 liposomes | |
| Dose (mg/kg) | 30 | 50 | 40 | 144 |
| Tumor AUC (μg · h/g) | 276 | 442 | 1744 | 7955 |
| Liver AUC (μg · h/g) | 10 | 37 | 1320 | 2838 |
| Spleen AUC (μg · h/g) | 77 | 162 | 402 | 3606 |
| Kidney AUC (μg · h/g) | 28 | 179 | 1164 | 2546 |

In the tumor, the docetaxel levels following administration of PEGylated TD-1 liposomes (expressed as a percent of the docetaxel level following administration of unecapsulated TD-1) increased after 3 to 7 days, particularly at the lower dose where the level reached 55% after 21 days. The ratio was generally stable in other tissues and ranged from around 1-2% in the liver and spleen up to 3-5% in the kidneys.

Levels of TD-1 in the liver, spleen, kidney, lung and skeletal muscle tissue appeared to fall into two categories (FIG. 5). The liver, spleen and kidney showed a pattern similar to the tumor with a slow uptake through the first 72 hours with concentrations slowly decreasing through the remainder of the 3 week period. The lung and skeletal muscle tissue contained the highest concentrations immediately after injection which decreased to concentrations close to the levels of detection after approximately 72 and 24 hours, respectively.

After approximately nine days, TD-1 concentrations in skeletal muscle tissue fell below the levels of quantitation for the 40 mg/kg dose of PEGylated TD-1 liposomes. A similar pattern of uptake and distribution for TD-1 occurred after the administration of PEGylated TD-1 liposomes at a dose of 144 mg/kg. After the high dose of PEGylated TD-1 liposomes, the lung and skeletal muscle tissue retained measurable concentrations of TD-1 throughout the observation period, but the concentrations tended to be lower than those found for the tumor, liver, spleen and kidney especially through the plateau period between 168 and 504 hours. The limits of quantitation of TD-1 were 0.5 μg/g for the liver, kidney, spleen and lung, and 2.0 μg/g for the skeletal muscle.

As for TD-1, uptake and elimination patterns fell into two categories for docetaxel derived from PEGylated TD-1 liposomes (FIG. 6). PEGylated TD-1 liposomes at doses of 40 or 144 mg/kg failed to produce quantifiable amounts of docetaxel in skeletal muscle tissue. The limits of quantitation for docetaxel were 0.5 μg/g for the liver, kidney, spleen and lung, and 1.0 μg/g for the skeletal muscle. The administration of docetaxel (50 mg/kg) distributed to the tissues for only a brief period of time. Concentrations of docetaxel fell below the limits of quantitation after 24 hours for most of the tissues except for the tumor which retained measurable levels of docetaxel through 216 hours (9 days).

### Example 4

### In Vivo Tumor Models, Comparative Results

A series of studies have been completed investigating the activity against various tumor cell lines implanted into immunodeficient mice and comparing the activity of PEGylated TD-1 liposomes with docetaxel. The studies were of broadly similar design. Tumor cell lines were implanted subcutaneously into the flank of nude (immunodeficient) mice and allowed to grow to a fixed size. Mice that did not grow tumors were rejected. Mice were allocated to receive either saline (control, included in all studies) or docetaxel or PEGylated TD-1 liposomes and administered the designated treatment by slow bolus intravenous injection. In each case, where possible, doses were selected as providing equivalent levels of toxicity/tolerance. The highest doses of TD-1 were usually limited by the volume that could be administered. Tumor volume was analyzed to determine tumor growth delay (TGD) and partial regression. Mice were removed from the study if they lost 20% of their initial bodyweight or became moribund or if their tumor volume exceeded 2500 mm³ or the tumor ulcerated. If less than half of the initial cohort of mice remained, that group was no longer graphed or included in further tumor analysis. However, any remaining animals were followed until completion of the in-life observation period and included in a survival analysis. The variable features of these studies are summarized in Table 13.

TABLE 13
Summary of variable features of In Vivo antitumor
activity studies in immunodeficient mice

| | | | Doses (mg | |
|---|---|---|---|---|
| | | | docetaxel/kg) ^{a} | |
| | | No./group, | | PEGylated |
| Tumor | Cell Line | sex | Docetaxel | TD-1 liposome |
| Head & neck | A253 | 10, female | 10, 20, 30 | 30, 60, 90 |
| Lung | A549 | 10, female | 10, 20, 30 | 30, 60, 90 |
| Lung | A549 | 10, female | 18, 27^{b} | 60, 90^{b} |
| Prostate | PC3 | 6, male | 9, 18, 27 ^{c} | 19, 38, 57^{d} |
| Breast | MDA-MB- | 8, female | 9, 18, 27 | 30, 60, 90 |
| | 435/PTK7 | | | |
| Fibrosarcoma | HT1080/ | 10, female | 9, 18, 27 | 30, 60, 90 |
| | PTK7 | | | |
| Epidermoid | A431 | 10, female | 20, 30 | 60, 90 |

^{a} Doses of PEGylated TD-1 liposomes are expressed as the docetaxel equivalent (dose of PEGylated TD-1 liposomes was 1.25 times greater).
^{b}Mice in Lung A549 efficacy study were given two doses, 21 days apart; all other studies were single dose investigations
^{c} Prostate PC3 Docetaxel (27 mg/kg) dose group had five mice.
^{d}Prostate PC3 efficacy study included 3 additional groups treated with non-PEGylated TD-1 at the same doses as PEGylated TD-1 liposomes.

All of the studies demonstrate that PEGylated TD-1 liposomes act as an active antitumor agent in these xenograft models, and possesses significantly greater antitumor activity compared to comparably tolerated doses of docetaxel.

Data from the study with A253 head & neck carcinoma model demonstrate that, compared with the saline control or docetaxel, administration of PEGylated TD-1 liposomes at 90 mg/kg resulted in a significant (p<0.05) reduction in tumor volume, inhibited tumor growth by 81% and increased tumor growth delay by 17 days (Table 14). There was a significant (p<0.05) increase in survival compared with control or docetaxel. Docetaxel failed to reduce A253 tumor volumes significantly or extend survival in mice. The antitumor response of PEGylated TD-1 liposomes occurred without observed toxicity. All animals tolerated the 80-day post-dosing observation period without apparent toxicity (weight loss) and there were no definitive treatment-related deaths observed during the experiment. Effects on tumor growth and survival are illustrated in FIG. 7.

TABLE 14
Efficacy parameters and survival in mice bearing
A253 xenograft tumors following treatment
with PEGylated TD-1 liposomes or docetaxel

| | | | | Median |
|---|---|---|---|---|
| Treatment and Dose | TGI (%)^{a} | TGD | TGD (%) | Survival (Days) |
| Control | — | — | 0 | 71 |
| Docetaxel (10 mg/kg) | 0 | 0 | 0 | 64 |
| Docetaxel (20 mg/kg) | 0 | 0 | 0 | 62 |
| Docetaxel (30 mg/kg) | 12 | 5 | 9 | 73 |
| PEGylated TD-1 | 0 | 0 | 0 | 58 |
| liposomes (30 mg/kg) | | | | |
| PEGylated TD-1 | 29 | 7 | 13 | 71 |
| liposomes (60 mg/kg) | | | | |
| PEGylated TD-1 | 81 | 17 | 31 | 84 |
| liposomes (90 mg/kg) | | | | |

^{a}Percent tumor growth inhibition (TGI %) calculated on day 31 days post treatment.

Data from the study with A549 non-small cell lung carcinoma (NSCLC) model demonstrate that, compared with the saline control or docetaxel, administration of PEGylated TD-1 liposomes at 90 mg/kg resulted in a significant reduction in tumor volume (p<0.05), inhibited tumor growth by 89% (Tumor Growth Inhibition, TGI, %) and caused partial tumor regression in 40% of animals (Table 15). In contrast, administration of docetaxel failed to reduce A549 tumor volumes significantly or extend survival in mice. The antitumor response of PEGylated TD-1 liposomes occurred without observed toxicity. All animals tolerated the 80-day post-dosing observation period without apparent toxicity (weight loss) and there were no definitive treatment-related deaths observed during the experiment. Effects on tumor growth and survival are illustrated in FIG. 8.

TABLE 15
Efficacy parameters and survival in mice bearing
A549 xenograft tumors following treatment
with PEGylated TD-1 liposomes or docetaxel

| | | Partial Tumor | Median |
|---|---|---|---|
| Treatment and Dose | TGI (%) | Regression (%) | Survival (Days) |
| Control | — | 20 | — |
| Docetaxel (10 mg/kg) | 0 | 0 | 96 |
| Docetaxel (20 mg/kg) | 4 | 0 | — |
| Docetaxel (30 mg/kg) | 38 | 10 | — |
| PEGylated TD-1 | 32 | 0 | — |
| liposomes (30 mg/kg) | | | |
| PEGylated TD-1 | 61 | 20 | — |
| liposomes (60 mg/kg) | | | |
| PEGylated TD-1 | 89 | 40 | — |
| liposomes (90 mg/kg) | | | |

Similar results were obtained with the same NSCLC model following two doses given 21 days apart. Administration of PEGylated TD-1 liposomes at 60 or 90 mg/kg resulted in significantly smaller tumor volumes compared to docetaxel at 18 or 27 mg/kg or with saline treated mice. While 18 and 27 mg/kg docetaxel also inhibited tumor growth, PEGylated TD-1 liposomes exhibited a greater antitumor effect as determined by TGD (Tumor Growth Delay) and partial tumor regression parameters (Table 16). PEGylated TD-1 liposomes increased survival at each dose evaluated compared to saline, and both 60 and 90 mg/kg dose levels increased median survival compared to all doses of docetaxel. Effects on tumor growth are illustrated in FIG. 9.

TABLE 16
Efficacy parameters and survival in mice bearing
A549 xenograft tumors following treatment
with docetaxel or PEGylated TD-1 liposomes

| | | | Partial Tumor | Median |
|---|---|---|---|---|
| | | | Regression | Survival |
| Treatment and Dose | TGD | TGD (%) | (%) | (Days) |
| Saline | — | — | 10 | 42 |
| Docetaxel (18 mg/kg) | 19 | 66 | 0 | 56 |
| Docetaxel (27 mg/kg) | 41 | 141 | 10 | 100 |
| PEGylated TD-1 | 66 | 228 | 70 | 112 |
| liposomes (60 mg/kg) | | | | |
| PEGylated TD-1 | —^{a} | —^{a} | 100 | 109 |
| liposomes (90 mg/kg) | | | | |

^{a}Tumors treated with PEGylated TD-1 liposomes (90 mg/kg) did not reach target size of 1 cm³, and were excluded from TGD and TGD %.

Data from the study with PC3 prostate tumor model demonstrate that PEGylated TD-1 liposomes possess antitumor activity greater than docetaxel when given at equitoxic doses. A single dose of PEGylated TD-1 liposomes (19, 38, or 57 mg/kg) caused a significant (p<0.05) reduction in tumor volume compared to saline treated mice. While 18 and 27 mg/kg docetaxel also inhibited tumor growth, PEGylated TD-1 liposomes exhibited greater antitumor effects as determined by TGD and partial tumor regression (Table 17). PEGylated TD-1 liposomes significantly (p<0.05) increased survival at each dose evaluated, and 57 mg/kg PEGylated TD-1 liposomes increased survival significantly (p<0.05) greater than all doses of docetaxel. Notably, the PEGylated TD-1 liposomes exhibited greater tumor volume inhibition than the non-PEGylated TD-1 liposomes. Treatment with PEGylated TD-1 liposomes at 19 mg/kg caused significantly smaller tumors than the equitoxic dose of docetaxel (9 mg/kg) and TD-1 liposomes (30 mg/kg), *, p<0.05. Effects on tumor growth and survival are illustrated in FIG. 10.

TABLE 17
Efficacy and survival parameters in mice bearing PC3
xenograft tumors following treatment with docetaxel,
TD-1 liposomes or PEGylated TD-1 liposomes

| | | | | Partial Tumor | Median |
|---|---|---|---|---|---|
| | | TGD | TGI | Regression | Survival |
| Treatment and Dose | TGD | (%) | (%) | (%) | (Days) |
| Saline | — | — | — | 0 | 35 |
| Docetaxel (9 mg/kg) | 11 | 42 | 38 | 0 | 47 |
| Docetaxel (18 mg/kg) | 41 | 154 | 91 | 33 | 81 |
| Docetaxel (27 mg/kg) | 42 | 157 | 98 | 60 | 84 |
| TD-1 liposomes | 21 | 78 | 53 | 17 | 57 |
| (30 mg/kg) | | | | | |
| TD-1 liposomes | 59 | 221 | 99 | 0 | 77 |
| (58 mg/kg) | | | | | |
| TD-1 liposomes | 62 | 233 | 101 | 50 | 104 |
| (88 mg/kg) | | | | | |
| PEGylated TD-1 | —^{a} | —^{a} | 80 | 17 | 56 |
| liposomes (19 mg/kg) | | | | | |
| PEGylated TD-1 | 66 | 250 | 100 | 67 | 89 |
| liposomes (38 mg/kg) | | | | | |
| PEGylated TD-1 | 71 | 268 | 101 | 83 | 126 |
| liposomes (57 mg/kg) | | | | | |

^{a}Tumors treated with 24 mg/kg PEGylated TD-1 liposomes did not reach a target size of 1 cm³,and were excluded from TGD and % TGD.

Data from the study with the MDA-MB-435/PTK7 human breast xenograft show that administration of a single dose of PEGylated TD-1 liposomes (30, 60, or 90 mg/kg) resulted in smaller median tumor volumes compared to saline. While 18 and 27 mg/kg docetaxel also inhibited tumor growth, PEGylated TD-1 liposomes exhibited a greater antitumor effect as determined by TGD, % TGI, and partial tumor regression parameters (Table 18). PEGylated TD-1 liposomes increased survival at each dose evaluated, and both 60 and 90 mg/kg PEGylated TD-1 liposomes increased survival compared to all doses of docetaxel. Effects on tumor growth and survival are illustrated in FIG. 11.

TABLE 18
Efficacy parameters and survival in mice bearing
MDA-MB-435/PTK7 xenograft tumors following treatment
with docetaxel or PEGylated TD-1 liposomes

| | | | | Partial Tumor | Median |
|---|---|---|---|---|---|
| | | TGD | TGI | Regression | Survival |
| Treatment and Dose | TGD | (%) | (%) | (%) | (Days) |
| Saline | — | — | — | 13 | 24 |
| Docetaxel (9 mg/kg) | 1 | 9 | — | 0 | 15 |
| Docetaxel (18 mg/kg) | 10 | 91 | 26 | 13 | 28 |
| Docetaxel (27 mg/kg) | 18 | 164 | 42 | 38 | 35 |
| PEGylated TD-1 | 15 | 137 | 60 | 25 | 35 |
| liposomes (30 mg/kg) | | | | | |
| PEGylated TD-1 | 28 | 255 | 98 | 25 | 49 |
| liposomes (60 mg/kg) | | | | | |
| PEGylated TD-1 | 22 | 200 | 97 | 50 | 44 |
| liposomes (90 mg/kg) | | | | | |

When tested against the HT1080/PTK7 human fibrosarcoma tumor, administration of a single dose of PEGylated TD-1 liposomes (30, 60, or 90 mg/kg) resulted in a significant (p<0.05) reduction in tumor volume compared to saline treated mice. While docetaxel (27 mg/kg) also inhibited tumor growth, PEGylated TD-1 liposomes exhibited a greater antitumor effect as determined by TGI, TGD and partial tumor regression parameters (Table 19). PEGylated TD-1 liposomes significantly (p<0.05) increased survival at each dose evaluated and increased median survival two to three fold over saline. In contrast, docetaxel did not significantly increase survival. Effects on tumor growth and survival are illustrated in FIG. 12.

TABLE 19
Efficacy parameters and survival in mice
bearing HT1080/PTK7 xenograft tumors following
treatment with docetaxel or PEGylated TD-1 liposomes

| | | | | Partial Tumor | Median |
|---|---|---|---|---|---|
| | | TGD | TGI | Regression | Survival |
| Treatment and Dose | TGD | (%) | (%) | (%) | (Days) |
| Saline | — | — | — | 20 | 15 |
| Docetaxel (9 mg/kg) | —^{a} | —^{a} | 52 | 0 | 11.5 |
| Docetaxel (18 mg/kg) | 7 | 70 | 31 | 10 | 23 |
| Docetaxel (27 mg/kg) | 16 | 160 | 89 | 70 | 30 |
| PEGylated TD-1 | 15 | 150 | 91 | 20 | 36 |
| liposomes (30 mg/kg) | | | | | |
| PEGylated TD-1 | —^{a} | —^{a} | 98 | 70 | 30 |
| liposomes (60 mg/kg) | | | | | |
| PEGylated TD-1 | 25 | 250 | 109 | 100 | 43 |
| liposomes (90 mg/kg) | | | | | |

^{a}Tumors treated with Docetaxel (9 mg/kg) and PEGylated TD-1 liposomes (60 mg/kg) did not reach target size of 1 cm³, and were excluded from TGD and TGD %.

Data from the study with A431 human epidermoid tumor xenografts shows that administration of a single dose of PEGylated TD-1 liposomes (60 or 90 mg/kg) resulted in a significant (p<0.05) reduction in tumor volume compared to saline treated animals. While 20 and 30 mg/kg docetaxel also inhibited tumor growth, PEGylated TD-1 liposomes exhibited a greater antitumor effect as determined by TGD and partial tumor regression (Table 20). Each treatment of PEGylated TD-1 liposomes (30, 60, or 90 mg/kg) significantly (p<0.05) increased survival greater than saline and all dose levels of docetaxel. Effects on tumor growth and survival are illustrated in FIG. 13.

TABLE 20
Efficacy parameters and survival in mice bearing
A431 xenograft tumors following treatment
with docetaxel or PEGylated TD-1 liposomes

| | | | | Partial Tumor | Median |
|---|---|---|---|---|---|
| | | TGD | TGI | Regression | Survival |
| Treatment and Dose | TGD | (%) | (%) | (%) | (Days) |
| Saline | — | — | — | 0 | 9 |
| Docetaxel (20 mg/kg) | 13 | 1408 | 123 | 63 | 17 |
| Docetaxel (30 mg/kg) | —^{a} | —^{a} | 120 | 50 | 9 |
| PEGylated TD-1 | 21 | 2268 | 92 | 25 | 26 |
| liposomes (60 mg/kg) | | | | | |
| PEGylated TD-1 | —^{a} | —^{a} | 110 | 75 | 53 |
| liposomes (90 mg/kg) | | | | | |

^{a}Tumors treated with Docetaxel (30 mg/kg) and PEGylated TD-1 liposomes (90 mg/kg) did not reach target size of 1 cm³, and were excluded from TGD and TGD %.

### Results of Lipid Composition Analysis:

The preparation of liposomal TD-1 (MP-3528) via the described remote loading technique has been evaluated for a series of lipid compositions. These compositions were chosen to evaluate the breadth of formulations which afforded encapsulated TD-1 while allowing for insertion of DSPE-PEG without significant loss or hydrolysis of TD-1. The methodology for preparation of these formulations can be summarized as follows:

1) Preparation of Vesicles Containing Encapsulated Ammonium Sulfate
a. Lipids were dissolved into alcohol (EtOH, which was then added to an aqueous solution of ammonium sulfate
b. The resultant vesicles were extruded to obtain a well-defined particle size
c. Diafiltration was performed to remove un-encapsulated ammonium sulfate

2) Remote Loading of MP-3528 into the Ammonium Sulfate Vesicles

3) Insertion of DSPE-PEG into Vesicles Containing the Remote Loaded MP-3528

4) Diafiltration Against a Histidine/Saline Buffer Solution

The lipids selected for this study encompassed a variety of characteristics including: differences in the chain length of di-alkyl-glycero-phosphatidyl cholines (C14-C18), unsaturation in the fatty acid of the di-alkyl-glycero-phosphatidyl cholines, variation on the mole % cholesterol in the mixture and the chain length of the PEG in the DSPE-PEG.

Successful preparation of liposomal formulations of TD-1 (MP-3528) were judged by:
1) Encapsulation of MP-3528, as measured by the ratio of Drug to total lipids. Higher values are indicative of higher levels of remote loading into the vesicles (values less than 0.1 indicate either less than optimal remote loading or loss of drug during the DSPE-PEG insertion step)
2) % of MP-3528 that had been released from the formulation (% free), with higher values of % free suggesting poor retention of drug (>25%)
3) % of Docetaxel, with low values indicating successful preparation without significant hydrolysis of the prodrug (>5%)
4) Particle size of the vesicles as an indication of vesicle integrity during processing (particle sizes greater than 120 nm suggest unacceptable changes during processing)
5) Incorporation of DSPE-PEG into the vesicles post-remote loading of MP-3528 (low values <1 mole % indicative of poor incorporation)

FIG. 14 provides a table of compositions evaluated.

### Results:

All formulations prepared that contained about 45% (molar) cholesterol or more resulted in compositions which satisfied the above criteria (Examples 1, 2, 5, 6, 7, 8, 10). The mixed unsaturated, saturated PCs (SOPC and POPC) gave acceptable results with respect to % free drug (Examples 11-13) as well as that containing the negatively charged DSPG (Example 16).

All formulations investigated with cholesterol levels at 25% (molar) gave compositions which failed in at least one of the above criteria (Examples 3, 18 and 21). In general, these formulations suffered from either inadequate drug incorporation (Examples 18 and 21) or % of drug which was “free” (Example 3), and in several cases with little to no incorporation of DSPE-PEG (Examples 18 and 21).

Intermediate cholesterol levels (35% molar) gave acceptable compositions with PCs that contained chain lengths of >C16, and where both chains were either saturated or unsaturated (Examples 4, 9, 14). In some examples, POPC, SOPC, DPPC and DMPC did not produce acceptable compositions (Examples 15, 17, 19 and 20). The POPC and SOPC examples (15 and 17) had unacceptable levels of “Free” drug while the DPPC and DMPC examples (19 and 20) did not contain adequate amount of drug.

Use of either DSPE-PEG(2000) or DSPE-PEG(5000) was shown to be acceptable (For DSPE-PEG(5000)—Example 8).

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A method for preparing a liposomal taxane, the method comprising:
a) forming a first liposome having a lipid bilayer comprising a phosphatidylcholine lipid and a sterol, wherein the lipid bilayer encapsulates an interior compartment comprising an aqueous solution;
b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoyl) group; and
c) forming a mixture comprising the loaded liposome and a poly(ethylene glycol)-phospholipid conjugate (PEG-lipid) under conditions sufficient to allow insertion of the PEG-lipid into the lipid bilayer;
thereby forming the liposomal taxane.

2. The method of claim 1, wherein the liposomal taxane has a drug to lipid ratio of from 0.12 to 0.25.

3. The method of claim 1, wherein the liposomal taxane has a drug to lipid ratio of from 0.14 to 0.19.

4. The method of claim 1, wherein the sterol present in the liposomal taxane is cholesterol, and is present in an amount of about 30% to 45% by weight relative to the amount of lipids.

5. The method of claim 1, wherein the sterol present in the liposomal taxane is cholesterol, and is present in an amount of about 40% to 45% by weight relative to the amount of lipids.

6. The method of claim 1, wherein the first liposome is formed from a lipid cholesterol combination selected from the group consisting of DSPC/DSPE/Chol, 45/10/45; DOPC/Chol, 55/45; DOPC/Chol, 65/35; HSPC/Chol, 55/45; DSPC/Chol, 55/45; DMPC/Chol, 55/45; DSPC/Chol, 65/35; DPPC/Chol, 55/45; SOPC/Chol, 55/45; POPC/Chol, 55/45; HSPC/Chol, 65/35; and wherein insertion of said PEG-lipid results in an amount of PEG-lipid of from about 1.9% to about 5.0% by weight relative to the combined abouts of lipid, cholesterol and PEG-lipid.

7. The method of claim 1, wherein the first liposome is formed from a lipid cholesterol combination selected from the group consisting of SOPC/Chol and POPC/Chol, wherein cholesterol is present in an amount of about 42-48 mol %, and wherein insertion of said PEG-lipid results in an amount of PEG-lipid of from about 1.9% to about 5.0% by weight relative to the combined amounts of lipid, cholesterol and PEG-lipid.

8. The method of claim 1, wherein the first liposome is formed from a lipid cholesterol combination selected from the group consisting of DOPC/Chol, HSPC/Chol, DSPC/Chol, and DPPC/Chol, wherein cholesterol is present in an amount of about 30-48 mol %, and wherein insertion of said PEG-lipid results in an amount of PEG-lipid of from about 1.9% to about 5.0% by weight relative to the combined abouts of lipid, cholesterol and PEG-lipid.

9. The method of claim 1, wherein the heterocyclyl-(C₂₋₅alkanoyl) group is selected from the group consisting of 5-(4-methylpiperazin-1-yl)-pentanoyl, 4-(4-methylpiperazin-1-yl)-butanoyl, 3-(4-methylpiperazin-1-yl)-propionoyl, 2-(4-methylpiperazin-1-yl)-ethanoyl, 5-morpholino-pentanoyl, 4-morpholino-butanoyl, 3-morpholino-propionoyl, 2-morpholino-ethanoyl, 5-(piperidin-1-yl)pentanoyl, 4-(piperidin-1-yl)butanoyl, 3-(piperidin-1-yl)propionoyl, and 2-(piperidin-1-yl)-ethanoyl.

10. The method of claim 1, wherein the heterocyclyl-(C₂₋₅alkanoyl) group is 4-(4-methylpiperazin-1-yl)-butanoyl.

11. The method of claim 1, wherein the phosphatidylcholine lipid is selected from the group consisting of dipalmitoylphosphatidylcholine (DPPC), distearoylphosphatidylcholine (DSPC), hydrogenated soy phosphatidylcholine (HSPC), and mixtures thereof; and wherein the sterol is cholesterol.

12. The method of claim 11, wherein the lipid bilayer comprises DSPC and cholesterol, and wherein the DSPC:cholesterol ratio is about 55:45 (mol:mol).

13. The method of claim 11, wherein the lipid bilayer comprises DSPC and cholesterol, and wherein the DSPC:cholesterol ratio is about 70:30 (mol:mol).

14. The method of claim 1, wherein the interior compartment of the first liposome comprises aqueous ammonium sulfate.

15. The method of claim 14, wherein loading the first liposome comprises forming an aqueous solution comprising the first liposome and the taxane, or a pharmaceutically acceptable salt thereof, under conditions sufficient to allow accumulation of the taxane in the interior compartment of the first liposome.

16. The method of claim 15, wherein step b) is conducted at a temperature of from about 50° C. to about 70° C.

17. The method of claim 15, wherein step b) is conducted such that the ratio of the combined weight of the phosphatidylcholine and the sterol to the weight of the taxane is about 1:0.01 to about 1:1.

18. The method of claim 17, wherein the ratio of the combined weight of the phosphatidylcholine and the sterol to the weight of the taxane is about 1:0.2.

19. The method of claim 1, wherein the PEG-lipid is a diacyl-phosphatidylethanolamine-N-[methoxy(polyethene glycol)].

20. The method of claim 19, wherein the PEG-lipid is selected from the group consisting of distearoyl-phosphatidylethanolamine-N-[methoxy(polyethene glycol)-2000] (DSPE-PEG2000) and distearoyl-phosphatidylethanolamine-N-[methoxy(polyethene glycol)-5000] (DSPE-PEG5000).

21. The method of claim 1 wherein step c) is conducted such that the ratio of the combined phosphatidylcholine and sterol to the PEG-lipid is from about 1000:1 (mol:mol) to about 20:1 (mol:mol).

22. The method of claim 21, wherein the ratio of the combined phosphatidylcholine and sterol to the PEG-lipid is from about 35:1 (mol:mol) to about 25:1 (mol:mol).

23. The method of claim 21, wherein the ratio of the combined phosphatidylcholine and sterol to the PEG-lipid is about 33:1 (mol:mol).

24. The method of claim 21, wherein the ratio of the combined phosphatidylcholine and sterol to the PEG-lipid is about 27:1 (mol:mol).

25. The method of claim 1, wherein step c) is conducted at a temperature of from about 35° C. to about 70° C.

26. The method of claim 25, wherein step c) is conducted at a temperature of from about 50° C. to about 55° C.

27. The method of claim 1, further comprising exchanging the liposomal taxane from the mixture in step c) to an aqueous solution that is substantially free of unencapsulated taxane and uninserted PEG-lipid.

28. The method of claim 1, further comprising lyophilizing the liposomal taxane.

29. A liposomal taxane prepared according to the method of claim 1.

30. A method for treating cancer, the method comprising administering to a subject in need thereof a liposomal taxane prepared according to the method of claim 1.

31-40. (canceled)
